(19) United States
(12) Patent Application Publication
(10) Pub. No.: US 2024/0218423 A1
(43) Pub. Date: Jul. 4, 2024

(54) USING DRIED SODIUM HYDROXIDE TO DENATURE DOUBLE STRANDED DNA

(71) Applicant: Illumina, Inc., San Diego, CA (US)

(72) Inventors: Eli Carrami, Cambridge (GB); Oliver Miller, Linton (GB); Sebastien Ricoult, Sawston (GB); Eilidh Rivers, Glasgow (GB); Dale Weekes, Haverhill (GB)

(73) Assignee: Illumina, Inc., San Diego, CA (US)

(21) Appl. No.: 18/390,990

(22) Filed: Dec. 20, 2023

Related U.S. Application Data
(60) Provisional application No. 63/476,432, filed on Dec. 21, 2022.

(51) Int. Cl. C12Q 1/6806; B01L 3/00

(52) U.S. Cl. CPC C12Q 1/6806; B01L 3/5082

(57) ABSTRACT

This application relates to methods of denaturing double-stranded DNA (dsDNA). In some examples, the methods utilize dried sodium hydroxide. In some examples, the method includes loading dsDNA into a first portion of a cartridge, wherein the second portion of the cartridge contains sodium hydroxide in a dry form; and mixing the dsDNA with the sodium hydroxide, thereby denaturing the dsDNA.

### CROSS REFERENCE TO RELATED APPLICATION

This application claims the benefit of U.S. Provisional Patent Application No. 63/476,432, filed Dec. 21, 2022 and entitled “Using Dried Sodium Hydroxide to Denature Double Stranded DNA,” the entire contents of which are incorporated by reference herein.

### FIELD

This application relates to methods of denaturing double-stranded DNA.

### BACKGROUND

In preparation for sequencing, DNA libraries often need to be denatured into single stranded molecules. Some current platforms use formamide at an elevated temperature to perform this denaturation step on-board. An alternative is to denature the DNA offboard by the user, with 0.1 M solution of sodium hydroxide.

Both of these strategies present issues. Formamide is a highly toxic substance and is carcinogenic. It therefore necessitates specialized disposal routes, hinders efforts to reach Corporate Social Responsibility goals, and is a main complaint from customers. In addition, denaturation using formamide requires a specialized heated compartment on the cartridge. Furthermore, due to its high viscosity, the mixing of the library with formamide further complicates the on-board fluidics systems. On the other hand, user-led denaturation by sodium hydroxide increases the hands-on time of sequencing, is a potential source of human error, and requires the customer to purchase and store additional chemicals and equipment. In addition, sodium hydroxide in solution is a strong corrosive agent. Therefore, new strategies for on-board library denaturation are needed.

### SUMMARY

Examples herein are related to methods, tubes, and cartridges that can be used to denature double-stranded DNA.

Some examples herein provide a method of denaturing double-stranded DNA (dsDNA), including loading the dsDNA into a first portion of a cartridge, wherein sodium hydroxide is located in a second portion of the cartridge, wherein the sodium hydroxide is in a dry form, and wherein the dsDNA and the dried sodium hydroxide are not in contact with each other, when the dsDNA is loaded into the first portion of the cartridge; and mixing the dsDNA with the sodium hydroxide, thereby denaturing the dsDNA.

In some examples, the method further includes neutralizing the sodium hydroxide.

In some examples, the method further includes hydrating the sodium hydroxide. In some examples, hydrating the sodium hydroxide is performed before mixing the dsDNA with the sodium hydroxide. In some examples, hydrating the sodium hydroxide includes adding water to the sodium hydroxide in the second portion of the cartridge, when the dsDNA and the sodium hydroxide are not in contact with each other. In some examples, mixing the dsDNA with the sodium hydroxide, hydrates the sodium hydroxide.

In some examples, the cartridge includes a constriction that temporarily inhibits contact between the dsDNA and the sodium hydroxide, when the dsDNA is loaded into the first portion of the cartridge. In some examples, suction is used to mix the dsDNA with the sodium hydroxide. In some examples, a syringe pump is used to create the suction.

In some examples, the cartridge includes an hourglass-shaped reservoir, and the first portion of the cartridge is an upper compartment of the hourglass-shaped reservoir, and the second portion of the cartridge is a lower compartment of the hourglass-shaped reservoir. In some examples, a constriction in the hourglass-shaped reservoir temporarily inhibits contact between the dsDNA and the sodium hydroxide, when the dsDNA is loaded into the first portion of the cartridge. In some examples, suction is used to mix the dsDNA with the sodium hydroxide. In some examples, the suction that is used to mix the dsDNA with the sodium hydroxide, hydrates the sodium hydroxide. In some examples, the method further includes hydrating the sodium hydroxide, and hydrating the sodium hydroxide precedes mixing the dsDNA with the sodium hydroxide.

In some examples, the cartridge includes a tube that connects the first portion of the cartridge to the second portion of the cartridge, and the method further includes transferring the dsDNA to the second portion of the cartridge, using the tube. In some examples, transferring the dsDNA to the second portion of the cartridge using the tube, mixes the dsDNA with the sodium hydroxide. In some examples, transferring the dsDNA to the second portion of the cartridge using the tube, hydrates the sodium hydroxide.

In some examples, the method further includes hydrating the sodium hydroxide, wherein hydrating the sodium hydroxide precedes transferring the dsDNA to the second portion of the cartridge, using the tube. In some examples, hydrating the sodium hydroxide includes water passing through the tube to contact the sodium hydroxide and the dsDNA.

Some examples herein provide a method of denaturing double-stranded DNA (dsDNA), including loading the dsDNA into an upper compartment of a spin tube that contains sodium hydroxide in a dry form, thereby denaturing the dsDNA, wherein a lower compartment of the spin tube contains a neutralizing agent; and centrifuging the spin tube to transfer the denatured DNA to the lower compartment of the spin tube, thereby neutralizing the sodium hydroxide.

In some examples, a timed reaction of between four (4) minutes and six (6) minutes denatures the dsDNA.

Some examples herein provide a method of denaturing double-stranded DNA (dsDNA), including loading dsDNA into a tube that contains sodium hydroxide in a dry form and a neutralizing agent including a time-triggered coat, thereby denaturing the dsDNA; and incubating the tube to allow for release of the neutralizing agent from the time-triggered coat, thereby neutralization the sodium hydroxide.

In some examples, the time-triggered coat includes microspheres. In some examples, the release of the neutralizing agent includes release of microspheres that contain the neutralizing agent.

Some examples herein provide a method of preparing a cartridge to denature double-stranded DNA (dsDNA), including loading sodium hydroxide into a second portion of the cartridge, wherein the sodium hydroxide is in a solution, wherein a first portion of the cartridge is configured to receive dsDNA; and drying the sodium hydroxide within the second portion of the cartridge.

In some examples, the method further includes adjusting the temperature during the drying step. In some examples, adjusting the temperature includes raising the temperature.

In some examples, the method further includes applying vacuum drying during the drying step.

In some examples, the method further includes applying freeze drying during the drying step.

In some examples, the drying step takes place in the presence of an inert gas.

In some examples, the solution includes an active agent. In some examples, the active agent includes betaine.

In some examples, the solution includes an inactive agent. In some examples, the inactive agent includes sodium chloride.

In some examples, the solution contains microspheres that contain the sodium hydroxide. In some examples, the microspheres include a coating. In some examples, the coating protects the microspheres from any one or more of moisture and carbon dioxide. In some examples, the coating reduces or prevents any static charge on the microspheres. In some examples, the coating is configured to allow for triggered release of the sodium hydroxide.

Some examples herein provide a method of preparing a cartridge to denature double-stranded DNA (dsDNA), including drying a solution containing sodium hydroxide on a porous structure; and loading the porous structure into a second portion of the cartridge, wherein a first portion of the cartridge is configured to receive dsDNA.

In some examples, the porous structure includes porous glass beads.

In some examples, the solution includes an active agent. In some examples, the active agent includes betaine.

In some examples, the solution includes an inactive agent. In some examples, the inactive agent includes sodium chloride.

In some examples, the method further includes dispensing the solution containing the sodium hydroxide onto the porous structure.

In some examples, the method further includes soaking the porous structure with the solution containing the sodium hydroxide.

In some examples, drying the solution containing sodium hydroxide on the porous structure includes adjusting the temperature. In some examples, adjusting the temperature includes raising the temperature.

In some examples, drying the solution containing sodium hydroxide on the porous structure includes applying vacuum drying.

In some examples, drying the solution containing sodium hydroxide on the porous structure includes applying freeze drying.

In some examples, the method further includes soaking the porous structure in the solution containing sodium hydroxide. In some examples, there is more than one soaking step and more than one drying step. In some examples, application of the more than one soaking step and the more one drying steps results in a multi-layered porous structure.

In some examples, the porous structure includes a coating. In some examples, the coating protects the porous structure from any one or more of moisture and carbon dioxide. In some examples, the coating reduces or prevents any static charge on the porous structure. In some examples, the coating allows for triggered release of the sodium hydroxide.

Some examples herein provide a spin tube, including a first compartment including sodium hydroxide; and a second compartment including a neutralizing agent.

In some examples, the first compartment is above the second compartment in the spin tube.

In some examples, the neutralizing agent is any one or more of sulfuric acid, phosphoric acid, carbon dioxide, nitric acid, and hydrochloric acid.

In some examples, the sodium hydroxide includes dried sodium hydroxide.

In some examples, the sodium hydroxide is in solution. In some examples, the concentration of the sodium hydroxide in solution is at a concentration between 0.1N and 1.6.N

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1A schematically illustrates an example of denaturing a dsDNA library using an hourglass-shaped reservoir in which a library is loaded in the upper compartment of the hourglass-shaped reservoir and dried sodium hydroxide is in the lower compartment of the hourglass-shaped reservoir. After loading, the library is transferred to bottom portion of the cartridge to allow a denaturation reaction to take place.

FIG. 1B schematically illustrates an example of denaturing a dsDNA library in which the dsDNA library is loaded into a first compartment of a cartridge that is connected via a tube to a second compartment of the cartridge that contains dried sodium hydroxide. After loading, the dsDNA library is transferred to the compartment containing the dried hydroxide to denature the dsDNA library.

FIG. 1C schematically illustrates an example of denaturing a dsDNA library in which dried sodium hydroxide is in a tubing system that is connected to a compartment that is loaded with the dsDNA library. After loading, water passes over and dissolves the sodium hydroxide as the water is transferred to the compartment containing the dsDNA library.

FIG. 2A schematically illustrates an example of denaturing and neutralizing a dsDNA library in which the dsDNA library is loaded into an upper compartment of a spin tube that contains dried sodium hydroxide. Neutralizing reagents are contained in the lower compartment of the spin tube. After denaturing the dsDNA library, centrifugation results in transfer of the dsDNA library to the lower compartment of the spin tube where the hydroxides are neutralized.

FIG. 2B schematically illustrates denaturing and neutralizing a library in which a dsDNA library is added to a spin tube that contains dried sodium hydroxide and neutralizing agent with a time-triggered coat. After a period of time, the dried sodium hydroxide denatures the dsDNA library followed by release of the neutralizing agent, resulting in neutralization of the hydroxides.

FIG. 3A shows results indicating that a final sodium hydroxide concentration between 0.8N and 1.6N results in a PF % that is equal to a standard workflow using sodium hydroxide and FIG. 3B shows that the GC % on the human genome had no bias of GC coverage.

FIG. 4 illustrates a scheme for denaturing a library in a single well.

FIG. 5 illustrates a scheme for denaturing a library in which two wells are used and the wells are separated by a membrane.

FIGS. 6 and 7 illustrate schematics of denaturation workflows.

FIG. 8A shows NaOH cakes in a desiccated environment in which the NaOH is uncoated.

FIG. 8B shows microspheres that contain a neutralizing agent.

FIGS. 9A and 9B show data that illustrates a time release delay of an active agent from microsphere coated with Opadry and a microsphere coated with double-coat of HPMC. FIG. 10 illustrates a schematic that compares a NovaSeqX denaturation workflow to a passive, simplified denaturation workflow.

FIGS. 11A and 11B illustrate an example process of a simplified, passive workflow for denaturing a library using a tube that contains sodium hydroxide and encapsulated, blocked microspheres. FIG. 11C provides data showing that sequencing metrics using the simplified, passive workflow compared to sequencing metrics using NovaSeq and NovaSeq x workflows.

FIG. 12 illustrates an example schematic of a simplified, passive sequencing workflow.

FIGS. 13A and 13B illustrate examples of different microspheres that contain different coatings, and the time it takes for rehydration.

FIG. 14 provides data showing sequencing metrics using microspheres that are single-encapsulated verse microspheres that are double-encapsulated.

FIGS. 15A-15E provide data showing primary sequencing metrics when a passive, simplified workflow is used to denature the DNA versus alternative workflows. FIGS. 16A-16C provide data showing secondary sequencing metrics when a passive, simplified workflow is used to denature DNA versus alternative workflows.

### DETAILED DESCRIPTION

Examples provided herein are methods and compositions that enable the use of precisely measured out solid sodium hydroxide as an alternative on-board library denaturant.

For example, cartridges are used that contain dried sodium hydroxide that is used as the denaturant. Unlike sodium hydroxide solution, which is a strong corrosive agent, dried sodium hydroxide is not corrosive. Thus, using and transporting cartridges that contain dried sodium hydroxide is much safer than if the cartridge contained sodium hydroxide solution.

Double-stranded DNA (dsDNA) can be loaded into the cartridge that contains the dried sodium hydroxide. After the dsDNA is loaded, the dried sodium hydroxide may be hydrated and mixed with the dsDNA, thereby denaturing the dsDNA. The method may further include neutralizing the sodium hydroxide. In some examples, the dsDNA is part of a dsDNA library.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the phrase “double-stranded DNA” refers to two (2) polynucleotide chains that are connected via hydrogen bonds. The phrase “double-stranded DNA” is used interchangeably with “dsDNA.”

As used herein, the phrase “GC” refers to guanine-cytosine. As used herein, the phrase “GC %” refers to the percentage of nitrogenous bases in a nucleotide molecule that are either guanine or cytosine.

As used herein, the term “cartridge” refers to any case or container capable of holding any solid or liquid material. As described herein, a “cartridge” can have a single compartment or portion, or it can have more than one (1) compartments or portions.

As used herein, the phrase “hourglass-shaped reservoir” refers to any case or container shaped like an hourglass and that is capable of holding any solid or liquid material. As described herein, an “hourglass-shaped reservoir” can have a single compartment or portion, or it can have more than one (1) compartments or portions.

As used herein, the phrase “spin tube” refers to any tube that is capable of being used in a centrifuge.

As used herein, the term “microsphere” refers to any hollow particle that has a circular or spherical shape. As described herein, a “microsphere” can range in size from 1 μM to 1,000 μM.

As used herein, the phrase “vacuum drying” refers to a drying method in which moisture in a substance is removed by placing the substance in an enclosed, air-tight container, and removing the moisture via a vacuum pump.

As used herein, the phrase “freeze drying” refers to a drying method in which moisture in a substance is removed by freezing the substance and then removing the ice on the substance that resulted from freezing the substance.

### Methods of Denaturing Double-Stranded DNA

Some examples herein provide a method of denaturing double-stranded DNA (dsDNA), including loading the dsDNA into a first portion of a cartridge, wherein sodium hydroxide is located in a second portion of the cartridge, wherein the sodium hydroxide is in a dry form, and wherein the dsDNA and the dried sodium hydroxide are not in contact with each other, when the dsDNA is loaded into the first portion of the cartridge; and mixing the dsDNA with the sodium hydroxide, thereby denaturing the dsDNA. In some examples, the dsDNA is part of a dsDNA library.

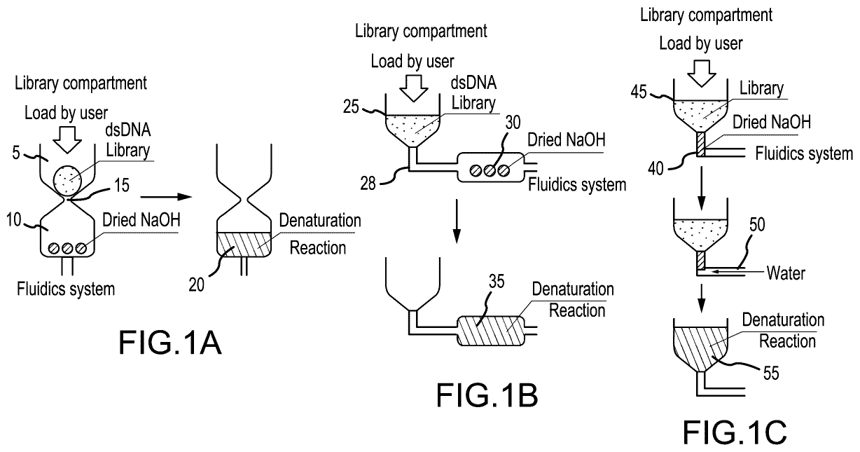

In some examples, the cartridge includes an hourglass-shaped reservoir, and the first portion of the cartridge is an upper compartment of the hourglass-shaped reservoir, and the second portion of the cartridge is a lower compartment of the hourglass-shaped reservoir. FIG. 1A schematically illustrates an example of denaturing a dsDNA library using an hourglass-shaped reservoir as the cartridge. The workflow shown in FIG. 1A shows a dsDNA library that is loaded in the upper compartment (5) of the hourglass-shaped reservoir and dried sodium hydroxide that is located the lower compartment (10) of the hourglass-shaped reservoir.

In some examples, the hourglass-shaped reservoir cartridge contains a constriction that temporarily inhibits contact between the dsDNA or the dsDNA library and the sodium hydroxide, when the dsDNA is loaded in the first portion of the cartridge. For example, as shown in FIG. 1A, prior to mixing the dsDNA library with the dried sodium hydroxide, the dsDNA library is not in contact with the dried sodium hydroxide due to a constriction (15) in the hourglass-shaped reservoir cartridge. In some examples, the constriction (15) temporarily avoids contact between the dsDNA and the dried sodium hydroxide via surface tension.

Mixing the dsDNA or the dsDNA library with the sodium hydroxide (15) denatures the dsDNA or the dsDNA library. For example, as shown in FIG. 1A, the dsDNA library is mixed with the dried sodium hydroxide (20) in the lower compartment of the hourglass-shaped reservoir. In some examples, suction is used to mix the dsDNA library with the dried sodium hydroxide. In some examples, a syringe pump is used to create the suction.

In some examples, the two (2) portions of the cartridge are two (2) separate compartments that are connected via a tube. A dsDNA or a dsDNA library can be loaded into a first compartment and dried sodium hydroxide is located in a second compartment. For example, FIG. 1B schematically illustrates an example of denaturing a dsDNA library in which the dsDNA library is loaded into a first compartment of a cartridge that is connected via a tube to a second compartment of the cartridge that contains dried sodium hydroxide. After loading the dsDNA library into the first compartment (25), the dsDNA library can be transferred to the compartment containing the dried sodium hydroxide (30) via a tube (28). This transfer mixes the dsDNA library with the dried sodium hydroxide (35) and denatures the dsDNA library.

In some examples, transferring the dsDNA to the second portion of the cartridge using the tube, mixes the dsDNA with the sodium hydroxide. In some examples, transferring the dsDNA to the second portion of the cartridge using the tube, hydrates the sodium hydroxide. In some examples, hydrating the sodium hydroxide precedes transferring the dsDNA to the second portion of the cartridge, using the tube.

In some examples, the dsDNA library is transferred to the compartment containing the dried sodium hydroxide using a fluidics system.

In some examples, the cartridge includes a first portion where dsDNA or a dsDNA library is loaded that is connected to a tube that contains the dried sodium hydroxide. For example, FIG. 1C schematically illustrates an example of denaturing a library in which dried sodium hydroxide is located within a tubing system (40) that is connected to a compartment loaded with a dsDNA library (45). In some examples, to initiate the denaturing reaction, after loading the dsDNA library, water passes over and dissolves the sodium hydroxide (50) as the water is transferred to the compartment containing the dsDNA library (55).

In some examples, for any of the dsDNA denaturing methods described herein, denaturing the dsDNA is performed at a pH equal to or greater than 8.5, for example, a pH of approximately 8.5, a pH of approximately 8.6, a pH of approximately 8.7, a pH of approximately 8.8, a pH of approximately 8.9, a pH of approximately 9.0, a pH of approximately 9.1, a pH of approximately 9.2, a pH of approximately 9.3, a pH of approximately 9.4, a pH of approximately 9.5, a pH of approximately 9.6, a pH of approximately 9.7, a pH of approximately 9.8, a pH of approximately 9.9, or a pH of approximately 10.0. In some examples, denaturing the dsDNA is performed at a pH greater than 10.0.

In some examples, denaturing the dsDNA is performed at a temperature between 20° ° C. and 22° C., for example, at approximately 20° C., at approximately 21° C., or at approximately 22ºC. In some examples, denaturing the dsDNA is performed at a temperature below 20° ° C. In some examples, denaturing the dsDNA is performed at a temperature above 20° C.

In some examples, any of the dsDNA denaturing methods described herein further include neutralizing the sodium hydroxide. In some examples, neutralizing the sodium hydroxide includes using a neutralizing agent. In some examples, the neutralizing agent is any one or more of sulfuric acid, phosphoric acid, carbon dioxide, nitric acid, and hydrochloric acid.

In some examples, any of the dsDNA denaturing methods described herein further include hydrating the sodium hydroxide. In some examples, the sodium hydroxide is hydrated in its final position such that the sodium hydroxide is not moved after it is hydrated. In some examples, hydrating the sodium hydroxide is performed before mixing the dsDNA with the sodium hydroxide.

In some examples, hydrating the sodium hydroxide includes adding water to the sodium hydroxide in the second portion of the cartridge, when the dsDNA and the sodium hydroxide are not in contact with each other.

In some examples, mixing the dsDNA with the sodium hydroxide, hydrates the sodium hydroxide.

In some examples, any of the dsDNA denaturing methods further include sequencing the dsDNA. In some examples, the sodium hydroxide is hydrated during sequencing.

In some examples, any of the dsDNA denaturing methods described herein use suction to mix the dsDNA with the sodium hydroxide. In some examples, the suction that is used to mix the dsDNA with the sodium hydroxide, hydrates the sodium hydroxide.

Some examples herein provide a method of denaturing dsDNA including loading the dsDNA into an upper compartment of a spin tube that contains sodium hydroxide in a dry form, thereby denaturing the dsDNA, wherein a lower compartment of the spin tube contains a neutralizing agent; and centrifuging the spin tube to transfer the denatured DNA to the lower compartment of the spin tube, thereby neutralizing the sodium hydroxide. In some examples, the dsDNA is part of a dsDNA library.

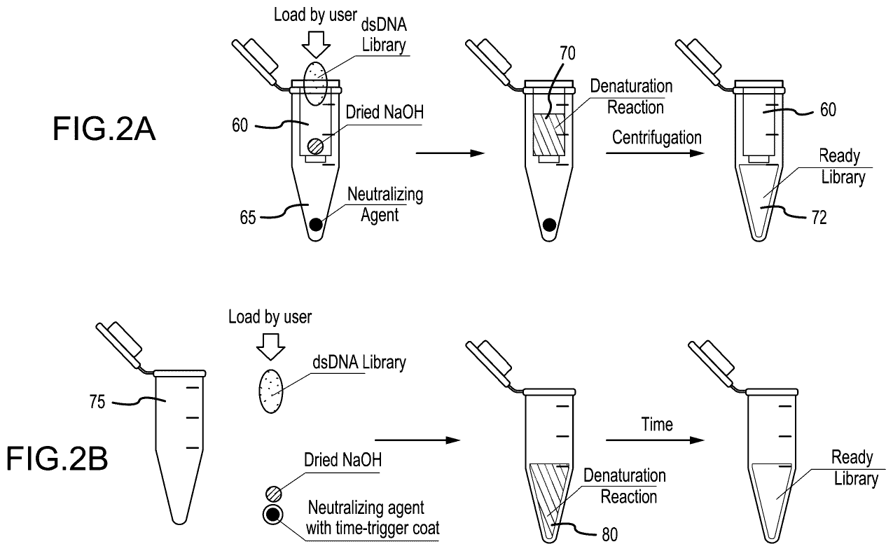

For example, FIG. 2A schematically illustrates an example of denaturing and neutralizing a dsDNA library in which the dsDNA library is loaded in the upper compartment of a spin tube that contains sodium hydroxide (60). Neutralizing reagents are located in the lower compartment of the spin tube (65). As shown in FIG. 2A, after loading the dsDNA library, the dsDNA library and the dried sodium hydroxide are mixed together (70) and a denaturation reaction denatures the dsDNA library. After the denaturation reaction, the spin tube (60) is centrifuged, which transfers to the dsDNA library to the lower compartment of the spin tube (72) containing the neutralizing agent, thereby neutralizing the sodium hydroxide.

In some examples, a timed reaction of between four (4) minutes and six (6) minutes denatures the dsDNA. In some examples, the timed reaction is approximately four (4) minutes, approximately five (5) minutes, or approximately six (6) minutes. In some examples, the timed reaction is less than four (4) minutes. In some examples, the timed reaction is greater than six (6) minutes.

Some examples herein provide a method of denaturing dsDNA, including loading dsDNA into a tube that contains sodium hydroxide in a dry form and a neutralizing agent including a time-triggered coat, thereby denaturing the dsDNA; and incubating the tube to allow for release of the neutralizing agent from the time-triggered coat, thereby neutralization the sodium hydroxide. In some examples, the dsDNA is part of a dsDNA library.

For example, FIG. 2B schematically illustrates denaturing and neutralizing a library in which a dsDNA library is added to a spin tube (75) that contains dried sodium hydroxide and neutralizing agent with a time-triggered coat. Adding these components together (80), initiates a denaturation reaction that denatures the dsDNA library. After incubating the tube, the neutralizing agent is released from the time-triggered coat, thereby neutralizing the sodium hydroxide.

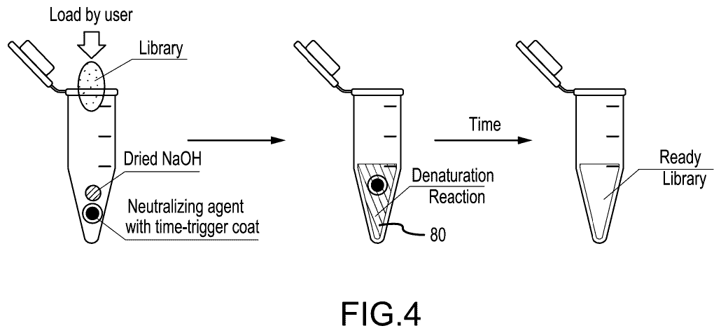

FIGS. 2B and 4 illustrate an example in which denaturing and neutralizing a library occurs in a single well using sodium hydroxide. The time triggered coat (shell) that covers the neutralizing agent and delays the release of the neutralizing agent such that the sodium hydroxide is not immediately neutralized upon incubation of the library with the sodium hydroxide. In some examples, the release of the neutralizing occurs after at least about 1 minute of incubation, for example, at least about 90 seconds, at least about 2 minutes, at least about 150 seconds, or at least about 3 minutes.

In some examples, the delay in release of the neutralizing agent incubation is linked to the thickness of the shell that covers the neutralizing agent. In some examples, the thicker the shell covering the neutralizing agent, the longer duration between the onset of incubation of the library with sodium hydroxide and the release of the neutralizing agent. Examples materials that can make up the shell covering the neutralizing agent include, but are not limited to, hydroxypropyl methylcellulose (HPMC), cellulose acetate, polyethylene glycol, PVP-co-PVAc, eudragits (e.g., eudragit RL and eudragit RS, isoleucine, Opadry CA, and polyester (e.g., co-polymer poly(lactic-co-glycolic acid (PLGA)).

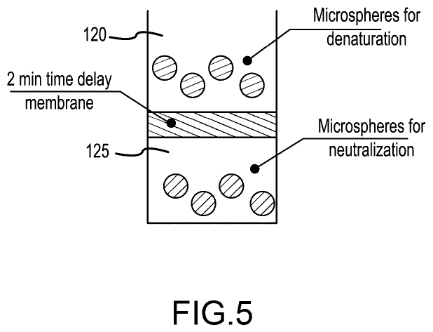

FIG. 5 illustrates an examples in which denaturing and neutralizing a library occurs in two wells that are separated by a time delay membrane. In the upper well 120, there are microspheres that include a denaturant (e.g., sodium hydroxide) that is used to denature the library. In the lower well 125, there are microspheres that contain one or more neutralizing agents.

In some examples, the any one or more neutralizing agents includes any one or more of sulfuric acid, phosphoric acid, carbon dioxide, nitric acid, and hydrochloric acid.

In some examples, the time-triggered coat includes microspheres. In some examples, release of the neutralizing agent includes release of microspheres that contain the neutralizing agent.

### Methods of Drying Sodium Hydroxide

Some examples herein provide a method of preparing a cartridge to denature double-stranded DNA (dsDNA), including loading sodium hydroxide into a second portion of the cartridge, wherein the sodium hydroxide is in a solution, wherein a first portion of the cartridge is configured to receive dsDNA; and drying the sodium hydroxide within the second portion of the cartridge.

In some examples, the concentration of the sodium hydroxide in the solution is between 0.1N and 1.6N. In some examples, the concentration of the sodium hydroxide in the solution is approximately 0.1N, approximately 0.2N, approximately 0.3N, approximately 0.4N, approximately 0.5N, approximately 0.6N, approximately 0.7N, approximately 0.8N, approximately 0.9N, approximately 1.0N, approximately 1.1N, approximately 1.2N, approximately 1.3N, approximately 1.4N, approximately 1.5N, or approximately 1.6N. In some examples, the concentration of the sodium hydroxide in the solution is less than 0.1N. In some examples, the concentration of the sodium hydroxide in the solution is greater than 1.6N.

In some examples, the method further includes adjusting the temperature during the drying step. In some examples, adjusting the temperature includes raising the temperature. In some examples, the temperature is raised to between 23° C. and 35° C., for example, to approximately 23° C., to approximately 24° C., to approximately 25° C., to approximately 26° C., to approximately 27° C., to approximately 28ºC, to approximately 29ºC, to approximately 30° C., to approximately 31° C., to approximately 32ºC, to approximately 33ºC, to approximately 34° C., or to approximately 35° C. In some examples, the temperature is raised to below 23ºC. In some examples, the temperature is raised to above 35ºC.

In some examples, the method further includes applying vacuum drying during the drying step. In some examples, the method further includes applying freeze drying during the drying step.

In some examples, the drying step takes place in the presence of an inert gas. In some examples, the inert gas is any one or more of helium, neon, argon, krypton, xenon, and radon.

In some examples, the solution includes an active agent. In some examples, the active agent includes betaine. In some examples, the solution includes an inactive agent. In some examples, the inactive agent includes sodium chloride.

In some examples, the solution contains microspheres that contain the sodium hydroxide. In some examples, the microspheres are made of glass or ceramic plastic. In some examples, the microspheres contain polymers.

In some examples, the microspheres range in size from between 1 μM and 1,000 μM. In some examples, the microspheres range in size from between 1 μM and 100 μM.

In some examples, the microspheres include a coating. In some examples, the coating is made up of a metal, such as, for example, iron, aluminum, magnesium, brass, zinc, or any other metal known in the art.

In some examples, the coating protects the microspheres from any one or more of moisture and carbon dioxide.

In some examples, the coating is configured to allow for triggered release of the sodium hydroxide.

Some examples herein provide a method of preparing a cartridge to denature double-stranded DNA (dsDNA), including drying a solution containing sodium hydroxide on a porous structure; and loading the porous structure into a second portion of the cartridge, wherein a first portion of the cartridge is configured to receive dsDNA.

In some examples, the porous structure includes porous beads. In some examples, the porous structure includes porous glass beads. In some examples, the porous structure includes porous polymer beads.

In some examples, the pore size on the porous structure is between 1.0 μm and 100 μm.

In some examples, the method further includes dispensing the solution containing the sodium hydroxide onto the porous structure.

In some examples, the method further includes soaking the porous structure with the solution containing the sodium hydroxide. In some examples, there is more than one (1) soaking step and more than one (1) drying step. In some examples, the more than one (1) soaking step includes two (2), three (3), four (4), five (5), six (6), seven (7), eight (8), nine (9), or ten (10) soaking steps. In some examples, the more than one (1) soaking step includes more than ten (10) soaking steps. In some examples, the more than one (1) drying step includes two (2), three (3), four (4), five (5), six (6), seven (7), eight (8), nine (9), or ten (10) drying steps. In some examples, the more than one (1) drying step includes more than ten (10) drying steps. In some examples, application of the more than one soaking step and the more than one drying step results in a multi-layered porous structure.

In some examples, the porous structure includes a coating. In some examples, the coating protects the porous structure from any one or more of moisture and carbon dioxide. In some examples, the coating reduces or prevents any static charge on the porous structure. In some examples, the coating allows for triggered release of the sodium hydroxide.

Spin Tubes Containing a Denaturant (e.g. Sodium Hydroxide) and a Neutralizing Agent

Some examples herein provide a spin tube that includes a first compartment including sodium hydroxide and a second compartment that includes a neutralizing agent.

In some examples, the first compartment is above the second compartment in the spin tube. In some examples, the first compartment and the second compartment are adjacent to each other in the spin tube.

In some examples, the first compartment is the upper compartment, the second compartment is the lower compartment, and the first compartment and the second compartment are separated by a membrane. In some embodiments, the first compartment comprises microspheres that contain a denaturant that functions to denature dsDNA. In some embodiments, a dsDNA library is added to the upper compartment. In some examples, the denaturant is sodium hydroxide. In some examples, the second compartment includes microspheres that contain a neutralizing agent that functions to neutralize the denaturant. In some examples, the membrane is a time-delay membrane that functions to delay contact between the microspheres in the lower compartment and the dsDNA library, after the dsDNA library is added to the upper compartment. In some examples, the time-delay membrane delays contact between the microspheres in the lower compartment and the dsDNA by at least 1 minute, by at least 90 seconds, by at least 2 minutes, by at least 150 seconds, or by at least 3 minutes.

In some examples, the denaturant includes sodium hydroxide. In some examples, the denaturant includes sodium chloride. In some examples, the denaturant includes trehalose.

In some examples, the neutralizing agent is any one or more of sulfuric acid, phosphoric acid, carbon dioxide, nitric acid, and hydrochloric acid.

In some examples, the sodium hydroxide is dried sodium hydroxide.

In some examples, the sodium hydroxide is in solution. In some examples, the concentration of the sodium hydroxide in solution is between 0.1N and 1.6N, for example, approximately 0.1N, approximately 0.2N, approximately 0.3N, approximately 0.4N, approximately 0.5N, approximately 0.6N, approximately 0.7N, approximately 0.8N, approximately 0.9N, approximately 1.0N, approximately 1.1N, approximately 1.2N, approximately 1.3N, approximately 1.4N, approximately 1.5N, or approximately 1.6N. In some examples, the concentration of the sodium hydroxide in solution is less than 0.1N. In some examples, the concentration of the sodium hydroxide in solution is greater than 1.6N.

In some examples, the sodium hydroxide in solution is dried in the spin tube. In some examples, the solution is dried through raising the temperature such that the temperature is between 23ºC and 35° C., for example, raising the temperature to approximately 23ºC, to approximately 24° C., to approximately 25° C., to approximately 26° C., to approximately 27° C., to approximately 28° C., to approximately 29ºC, to approximately 30° C., to approximately 31° C., to approximately 32ºC, to approximately 33ºC, to approximately 34ºC, or to approximately 35ºC. In some examples, the temperature is raised to below 23° C. In some examples, the temperature is raised to above 35° C.

In some examples, the sodium hydroxide is dried through applying vacuum. In some examples, the sodium hydroxide is dried through applying freeze drying.

In some examples, the drying the sodium hydroxide step takes place in the presence of an inert gas. In some examples, the inert gas is any one or more of helium, neon, argon, krypton, xenon, and radon.

Simplified, Passive Workflow for Denaturing a dsDNA Library

Some examples provided herein relate to denaturing dsDNA using a simplified, passive workflow. In some examples, the simplified, passive workflow includes a step of denaturing DNA using a denaturant and a step of neutralizing the denaturant.

In some examples, the simplified, passive workflow includes a tube that includes a first type of microsphere and a second type of microsphere. In some examples, a dsDNA library is added to the tube. In some examples, the first type of microsphere includes a denaturant. In some examples, the denaturant includes any one or more of sodium hydroxide, sodium chloride, and trehalose. In some examples, the second type of microsphere includes a neutralizing agent (blocking agent). In some examples, the denaturant includes any of the denaturants disclosed in Table 2.

In some examples, the second type of microsphere includes at least one shell that encapsulates the microsphere. In some examples, the at least one shell is made up of any one or more of hydroxypropyl methylcellulose (HPMC), cellulose acetate, polyethylene glycol, PVP-co-PVAc, eudragits (e.g., eudragit RL and eudragit RS, isoleucine, Opadry CA, and polyester (e.g., co-polymer poly(lactic-co-glycolic acid (PLGA)). In some examples, the at least one shell comprises two shells that encapsulate the microsphere. In some examples, the two shells are made up of any one or more of hydroxypropyl methylcellulose (HPMC), cellulose acetate, polyethylene glycol, PVP-co-PVAc, eudragits (e.g., eudragit RL and eudragit RS, isoleucine, Opadry CA, and polyester (e.g., co-polymer poly(lactic-co-glycolic acid (PLGA)). In some examples, the two shells includes Shell 1 and Shell 2, as disclosed in Table 4.

In some examples, the second type of microsphere includes a neutralizing agent. In some examples, the neutralizing agent includes any one or more of sulfuric acid, phosphoric acid, carbon dioxide, nitric acid, and hydrochloric acid. In some examples, the neutralizing agent includes the neutralizing agent disclosed in Table 3 (neutrablock).

In some examples, the at least one or more shells of the second microsphere delays the release of the neutralizing agent, after the dsDNA is added to the tube. In some examples, the delay of the release of the neutralizing agent after the dsDNA library is added to the tube is at least 1 minute, at least 90 seconds, at least 2 minutes, at least 150 seconds, or at least 3 minutes.

In some examples, the pH is adjusted in the tube during the simplified, passive workflow. In some examples, the pH is between about 6 and about 9 during the step of denaturing the dsDNA library, for example, a pH of about 7 or a pH of about 8. In some examples, the pH is between about 12 and 14 during the step of neutralizing the denaturant, for example, a pH of about 14.

### Working Examples

The following examples are intended to be purely illustrative, and not limiting of the present disclosure.

### Example 1. Dried Sodium Hydroxide is Capable of Denaturing dsDNA

Proof of concept experiments were performed to show the feasibility of using dried sodium hydroxide at various concentrations to denature dsDNA libraries.

Various concentrations of sodium hydroxide solution were left to dry overnight in 5 ul volume (The various concentrations of the sodium hydroxide that were used are shown in Table 1). The following day, the dsDNA library was directly used to hydrate the sodium hydroxide and initiate the denaturation reaction. The libraries were sequenced for a single 150 cycles read on a HiSeq X instrument (Illumina).

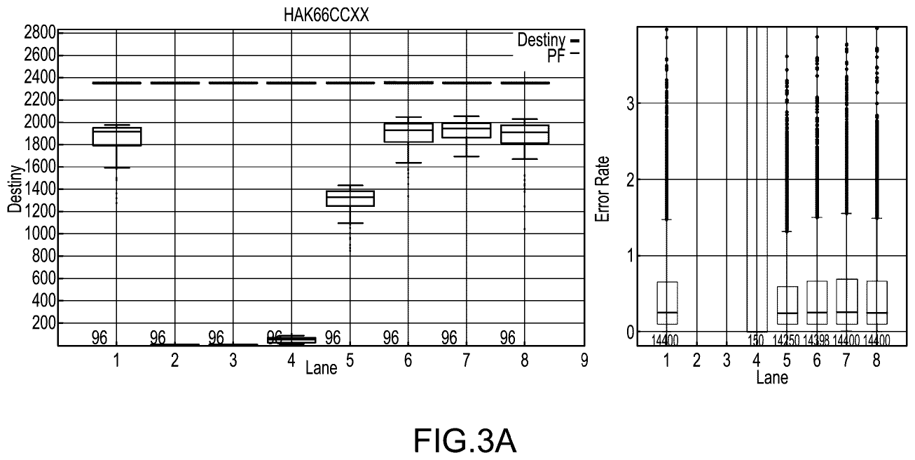

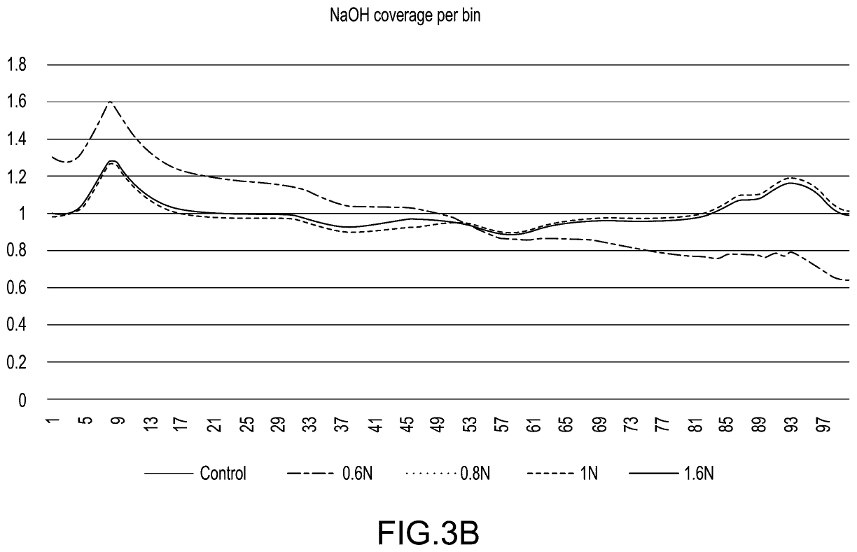

The data showed that a wide range of sodium hydroxide concentrations from at 0.8N to 1.6N resulted in PF % equal to a standard workflow using sodium hydroxide solution, without any impact on error rate (see FIG. 3A). Moreover analysis of the coverage over GC % on the human genome showed no bias of GC coverage (see FIG. 3B).

TABLE 1

| Lane | Condition | Dried? |
|---|---|---|
| 1 | Standard | No |
| 2 | 0.1N | Yes |
| 3 | 0.2N | Yes |
| 4 | 0.4N | Yes |
| 5 | 0.6N | Yes |
| 6 | 0.8N | Yes |
| 7 | 1.0N | Yes |
| 8 | 1.6N | Yes |

### Example 2. Denaturation Workflows

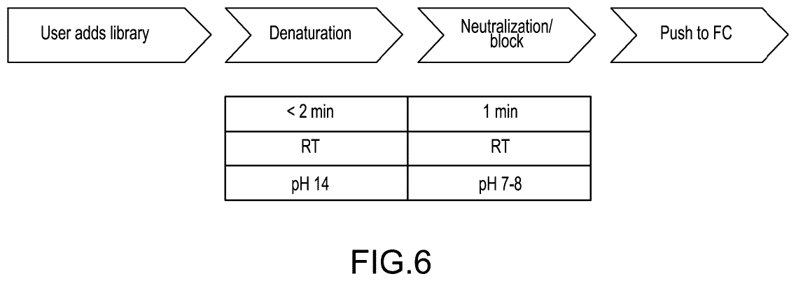

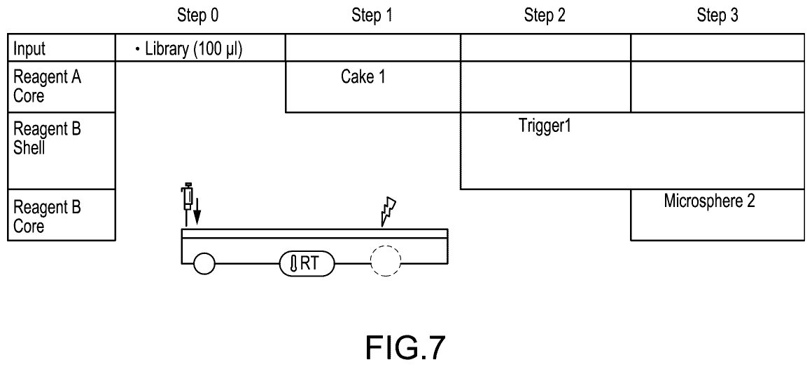

A simplified, passive denaturation workflow is illustrated in FIG. 6. The user adds the library to a tube, which is followed to denaturation of the library. The denaturant used to denature the library is then blocked. The library can then be sequenced using conventional sequencing techniques. FIG. 7 illustrates a passive library denaturation and a neutralizing/blocking workflow. In Step 0, the user adds the library to the tube. The library comes into contact with cake 1 (e.g., a microsphere containing a sodium hydroxide). There is then a trigger that causes a delay in the neutralization of the sodium hydroxide. As described below, this trigger that causes the delay in neutralization can either be a time-triggered coat that encapsulates a microsphere or time-delay membrane.

There at least two ways this process can be implemented, in a tube. FIG. 4 illustrates a first implementation. In this example, a library is added to a tube that contains a single well. In the tube, the library comes into contact with sodium hydroxide which denatures the library. A neutralizing agent is then released to neutralize the sodium hydroxide. The neutralizing agent is contained within a microsphere that contains a time-triggered coat. As a result, there is a delay between the time the library is initially incubated in the tube and the release of the neutralizing agent. The delay can be anywhere between 1 minute and 3 minutes. FIG. 5 illustrates a second implementation. In this examples, a library is added to a tube that contains two wells, and the wells are separated by a time delay membrane. Initially, the library contacts microspheres containing sodium hydroxide in the upper well 120. After a time delay (between 1 and 3 minutes), due to the time-delay membrane, the sodium hydroxide is neutralized.

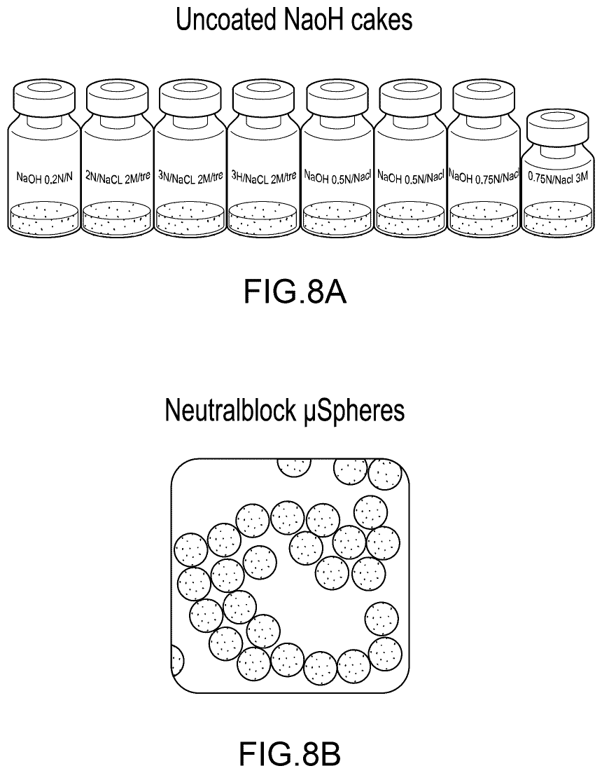

FIGS. 8A and 8B illustrate examples of reagent formulations for use in the simplified, passive denaturation workflow. As shown in FIG. 8A, when the sodium hydroxide cakes are in tubes in a desiccated environment, the cakes do not need to be coated. As shown in FIG. 8B, the microspheres contain a blocking formulation (neutralblock). An example of a blocking formulation includes:
1.334 nM blocker oligos
1 M MOPS
10 mM MgCl₂
17.4% trehalose
0.2% polysorbate 20

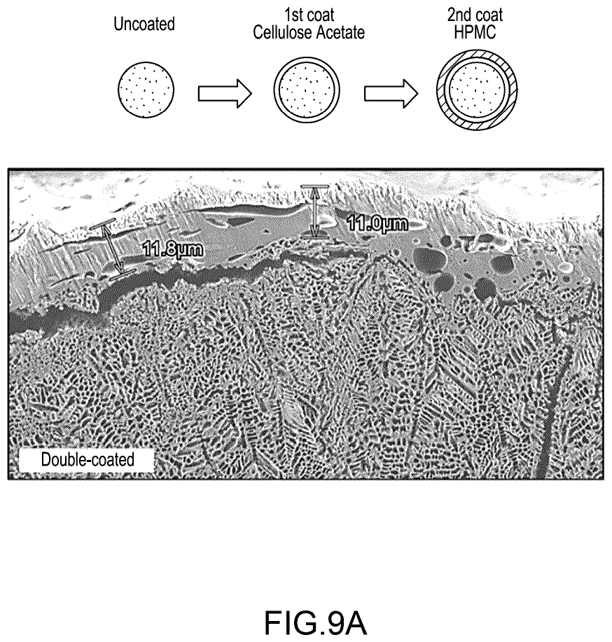

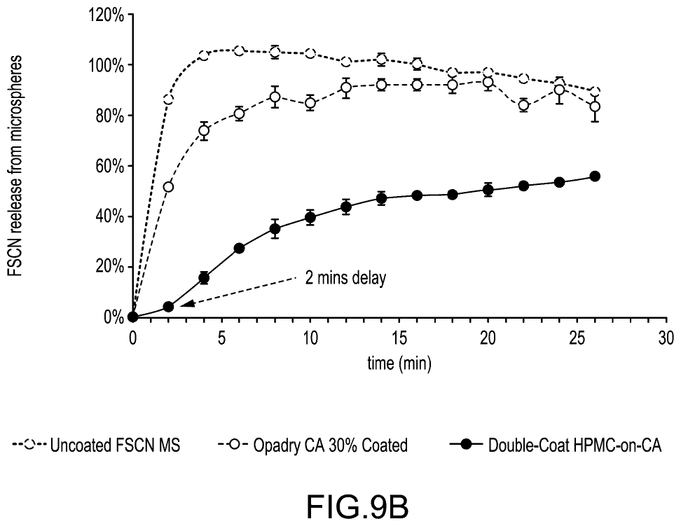

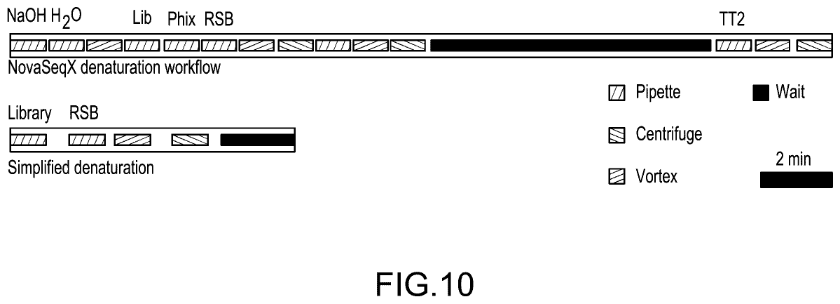

FIGS. 9A and 9B show data of a 2 minute delayed release of microspheres single-coated with cellulose acetate and microspheres double-coated with cellulose acetate and HPMC. The data show that the single-coated and double-coated microspheres delayed the release of the neutralizing agent relative to the uncoated, control microspheres.

### Lyophilized Formulations for Simplified, Passive Denaturation

Table 2 provides examples of denaturants that can be used in a simplified, passive workflow.

TABLE 2
Denaturants

| Denaturant | Conc. | Units | MW | Mg per ml |
|---|---|---|---|---|
| NaOH | 500 | mM | 39.997 | 19.9985 |
| NaCl | 2000 | mM | 58.44 | 116.8800 |
| Trehalose | 185 | mM | 342.296 | 63.3248 |

| | |
|---|---|
| Total mass | 200.20 |
| Total solute content (%) | 20.02 |

Table 3 provides an example of a blocking formulation (neutralizing formulation) (Neutrablock)

TABLE 3
Neutrablock

| Neutrablock | Conc. | Units | MW | Mg per ml |
|---|---|---|---|---|
| Blocker oligos | 0.1334 | mM | | 0.0000 |
| MOPS | 1000 | mM | 209.2633 | 209.2633 |
| MgCl₂ | 10 | mM | 95.211 | 0.9521 |
| Polysorbate 20 | 0.2 | % | 1,227.54 | 2.0000 |
| trehalose | 700 | mM | 342.296 | 239.6072 |

| | |
|---|---|
| Total mass | 451.82 |
| Total solute content (%) | 45.18 |

Table 4 provides embodiments of formulations of shells that can be used to encapsulate microspheres.

TABLE 4

| Encapsulation | % | Components (+Efka & Makkon) |
|---|---|---|
| Shell 1 | 20 | Thymol blue, cellulose acetate 50K |
| Shell 2 | 27 | HPMC E5, Mg Stearate (1.23%) |

### Comparing the Simplified, Passive Workflow to a Typical Denaturation Process

Using the simplified, passive denaturation workflow results in a simplified user experience relative to a typical denaturation process as shown in Table 5 below and in FIG. 10.

TABLE 5

| | Typical denaturation | Simplified, passive |
|---|---|---|
| | process | denaturation |
| Touch points | 14 | 5 |
| TAT (min) | 15 | 5 |
| HOT (min) | 5 | <0.5 |
| Part count | 6 | 1 |

### Adjusting the pH in the Simplified, Passive Workflow

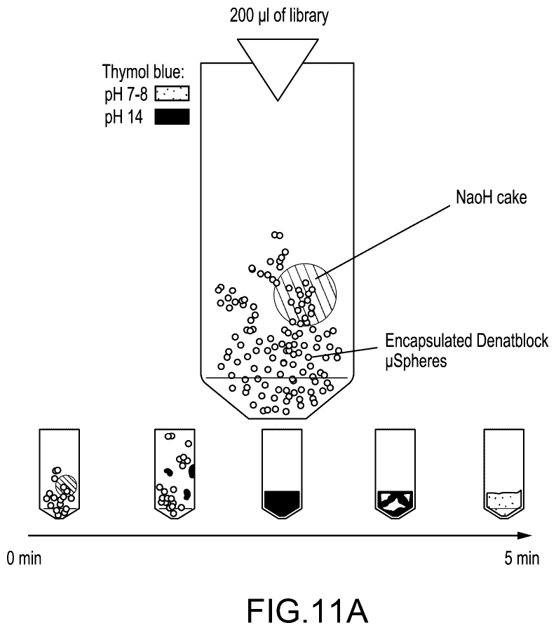

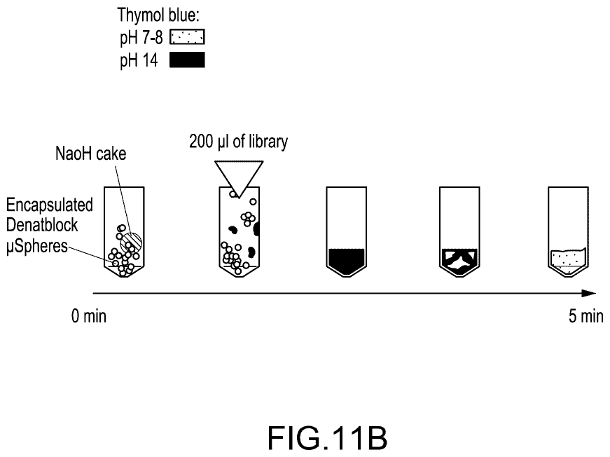

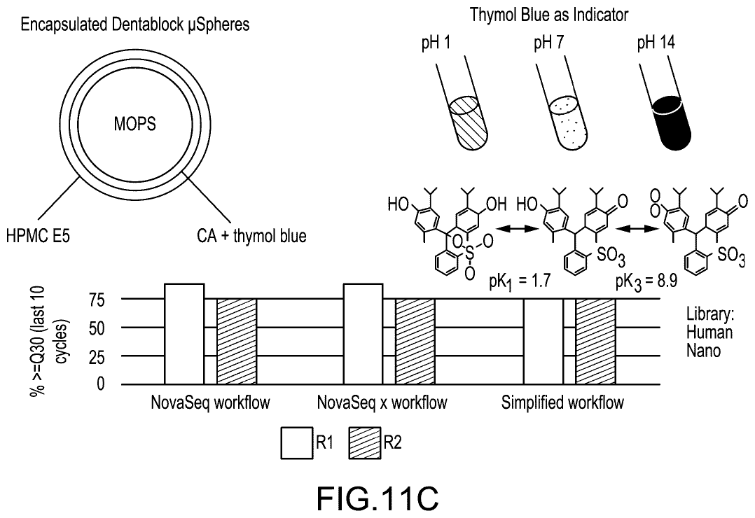

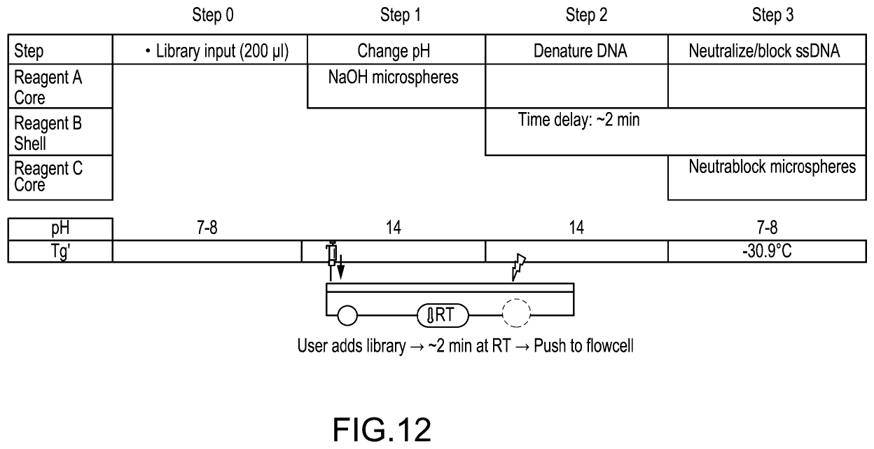

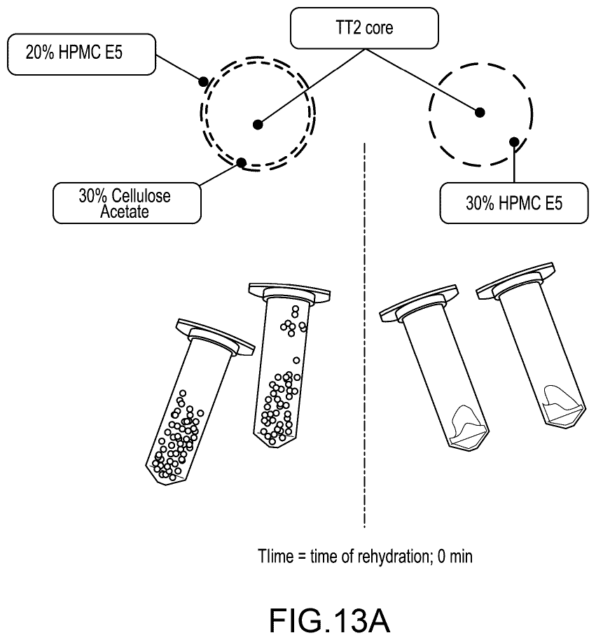

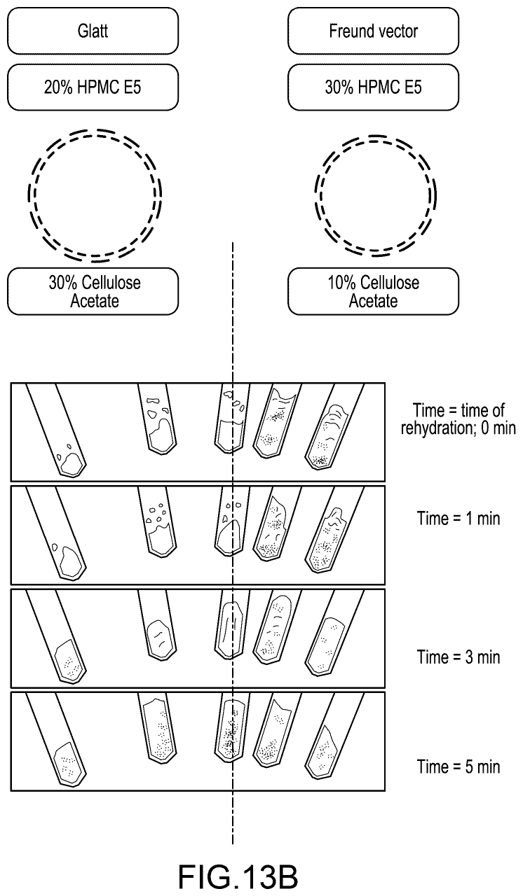

Adjusting the pH from 14 to 7 yields a sequencing-ready and compatible denaturing library. FIG. 12 illustrates a schematic of a simplified, passive workflow in which the pH is adjusted at different time steps in the workflow. When the library is added to the tube (Step 0), the pH is between 7 and 8. When the library comes into contact with sodium hydroxide microspheres (Step 1) and during denaturation (Step 2), the pH is at 14. When the neutralizing agent is released (Step 3), the pH is reduced back to between 7 and 8. FIGS. 11A and 11B illustrate schematics in which a library is added to a tube as part of a simplified, passive workflow. As shown in FIGS. 11A and 11B, based on the use of thymol blue, the pH changes from 14 (when the library comes into contact with the sodium hydroxide cakes and the library is denatured) to 7, when the sodium hydroxide is neutralized. FIG. 11C provides data showing that secondary sequencing metrics using this workflow were comparable to typical NovaSeq and NovaSeq x workflows. FIG. 13A illustrates examples of two different microspheres that contain different coatings, and the time it takes for rehydration. FIG. 13B illustrates examples of microspheres coated with a different percentages of cellulose acetate, and the time it takes for rehydration.

Comparing Sequence Metrics between Single and Double Encapsulation

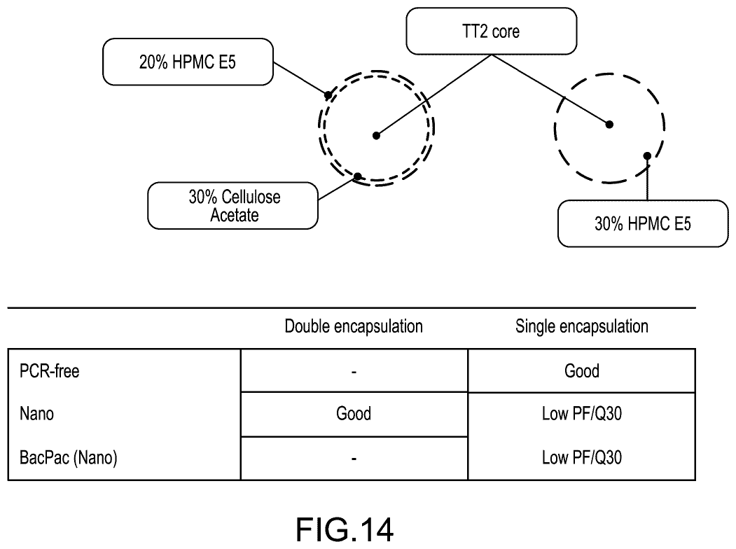

Sequencing metrics were compared in simplified, passive workflows between singled-encapsulated and double-encapsulated microspheres. As shown in FIG. 14, the double-encapsulated microsphere included a first outer layer (shell) of 30% cellulose acetate and a second outer layer (shell) of 20% HPMC, and the single-capsulated microsphere included an outer layer (shell) of 30% HPMC. The data in FIG. 14 shows that the single-encapsulated microsphere had inferior primary sequencing metrics compared to the double-encapsulated microsphere.

### Analyzing Primary and Secondary Sequencing Metrics Using the Simplified, Passive Workflow

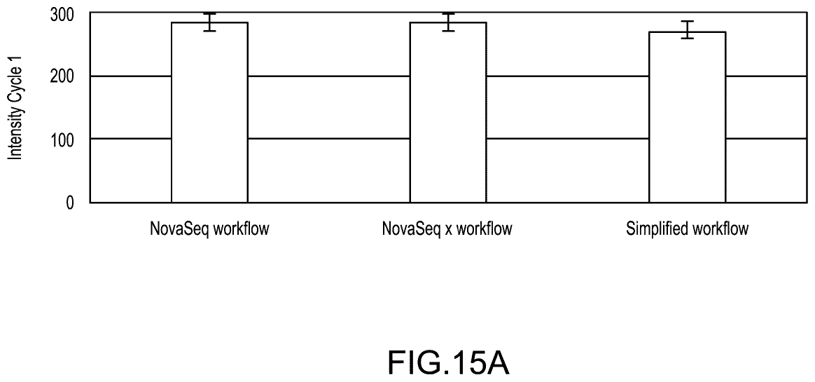

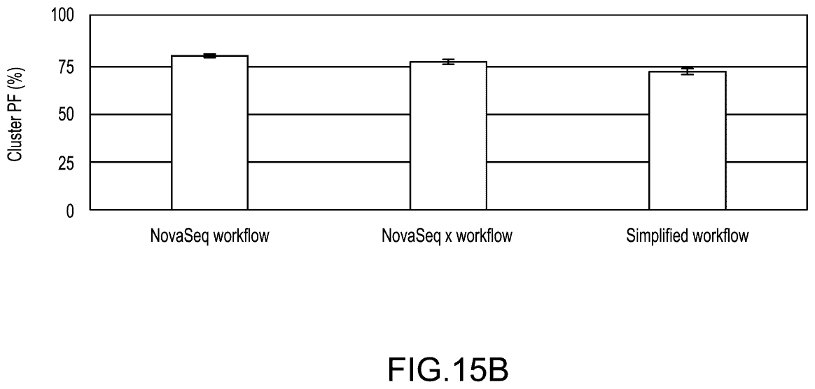

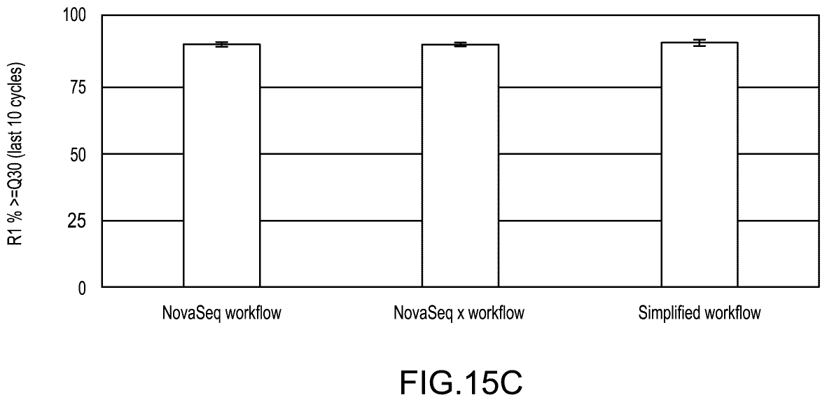

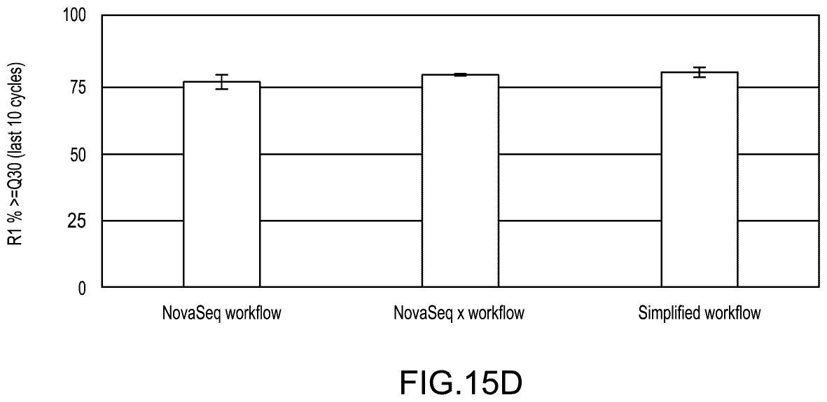

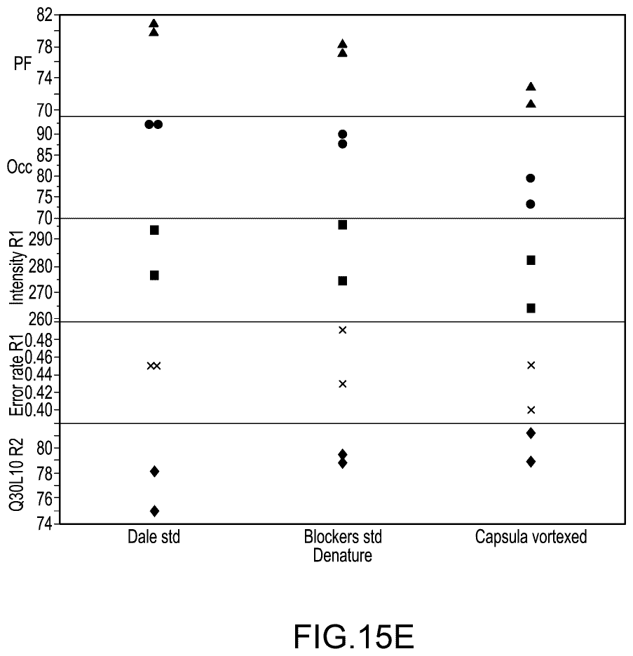

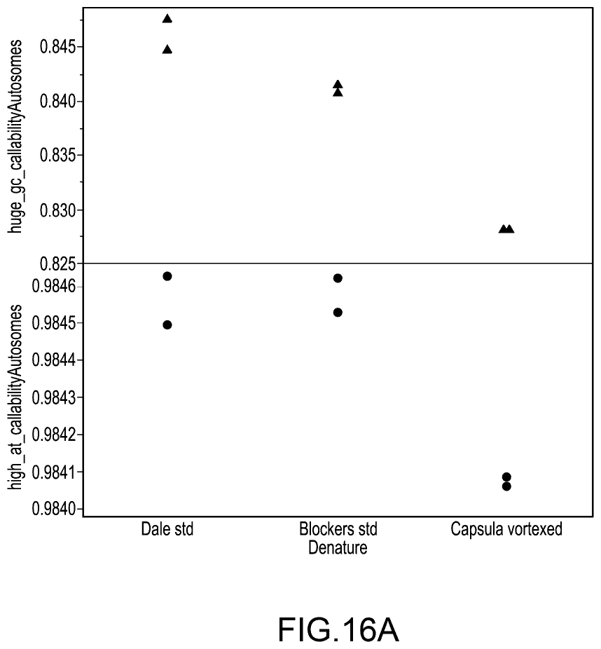

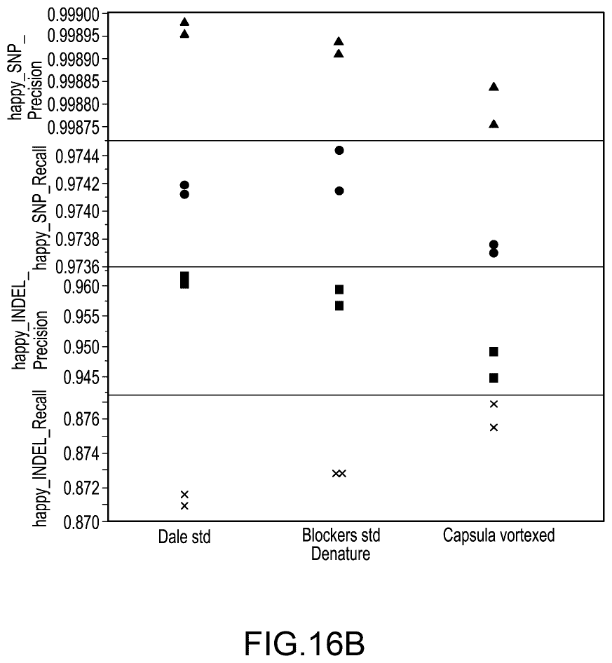

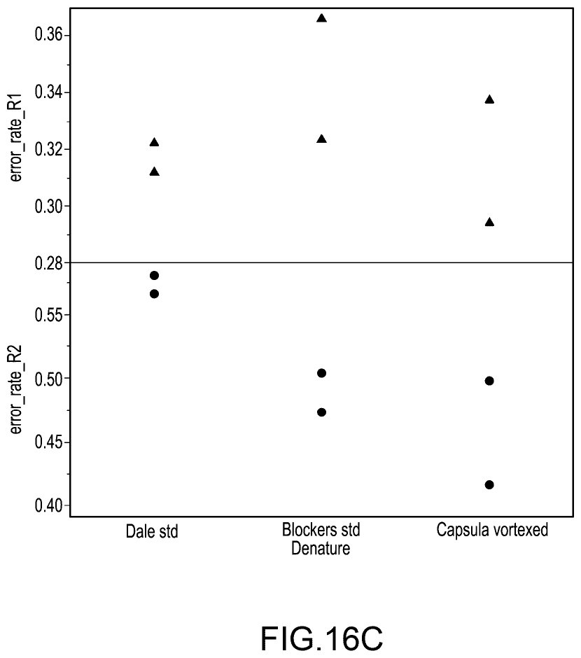

FIGS. 15A-15E provide data showing that sequencing libraries using the simplified, passive workflow to denature DNA (“Simplified workflow” and “Capsula vortexed”) resulted in primary sequencing metrics comparable to workflows in which DNA is denatured through alternative workflows (“NovaSeq workflow” and “NovaSeq x workflow”). FIGS. 16A-16C provide data showing that sequencing libraries using the simplified, passive workflow to denature DNA (“Capsula vortexed”) resulted in error rates that are comparable to workflows in which DNA is denatured through alternative workflows (“Dale std” and Blockers std Denature”).

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the examples provided herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of denaturing double-stranded DNA (dsDNA), comprising:
loading the dsDNA into a first portion of a cartridge, wherein sodium hydroxide is located in a second portion of the cartridge, wherein the sodium hydroxide is in a dry form, and
wherein the dsDNA and the dried sodium hydroxide are not in contact with each other, when the dsDNA is loaded into the first portion of the cartridge; and
mixing the dsDNA with the sodium hydroxide, thereby denaturing the dsDNA.

2. The method of claim 1, further comprising neutralizing the sodium hydroxide.

3. The method of claim 1, further comprising hydrating the sodium hydroxide.

4. The method of claim 3, wherein hydrating the sodium hydroxide is performed before mixing the dsDNA with the sodium hydroxide.

5. The method of claim 4, wherein hydrating the sodium hydroxide comprises adding water to the sodium hydroxide in the second portion of the cartridge, when the dsDNA and the sodium hydroxide are not in contact with each other.

6. The method of claim 3, wherein mixing the dsDNA with the sodium hydroxide, hydrates the sodium hydroxide.

7. The method of claim 1, wherein the cartridge comprises a constriction that temporarily inhibits contact between the dsDNA and the sodium hydroxide, when the dsDNA is loaded into the first portion of the cartridge.

8. The method of claim 7, wherein suction is used to mix the dsDNA with the sodium hydroxide.

9. (canceled)

10. The method of claim 1, wherein the cartridge comprises an hourglass-shaped reservoir, and wherein the first portion of the cartridge is an upper compartment of the hourglass-shaped reservoir, and the second portion of the cartridge is a lower compartment of the hourglass-shaped reservoir.

11. The method of claim 10, wherein a constriction in the hourglass-shaped reservoir temporarily inhibits contact between the dsDNA and the sodium hydroxide, when the dsDNA is loaded into the first portion of the cartridge.

12. The method of claim 11, wherein suction is used to mix the dsDNA with the sodium hydroxide.

13. The method of claim 12, wherein the suction that is used to mix the dsDNA with the sodium hydroxide, hydrates the sodium hydroxide.

14. The method of claim 10, further comprising hydrating the sodium hydroxide, wherein hydrating the sodium hydroxide precedes mixing the dsDNA with the sodium hydroxide.

15. The method of claim 1, wherein the cartridge comprises a tube that connects the first portion of the cartridge to the second portion of the cartridge, the method further comprising transferring the dsDNA to the second portion of the cartridge, using the tube.

16. The method of claim 15, wherein transferring the dsDNA to the second portion of the cartridge using the tube, mixes the dsDNA with the sodium hydroxide.

17. The method of claim 15, wherein transferring the dsDNA to the second portion of the cartridge using the tube, hydrates the sodium hydroxide.

18. The method of claim 15, further comprising hydrating the sodium hydroxide, wherein hydrating the sodium hydroxide precedes transferring the dsDNA to the second portion of the cartridge, using the tube.

19. The method of claim 18, wherein hydrating the sodium hydroxide comprises water passing through the tube to contact the sodium hydroxide and the dsDNA.

20. A method of denaturing double-stranded DNA (dsDNA), comprising:
loading the dsDNA into an upper compartment of a spin tube that contains sodium hydroxide in a dry form, thereby denaturing the dsDNA,
wherein a lower compartment of the spin tube contains a neutralizing agent; and
centrifuging the spin tube to transfer the denatured DNA to the lower compartment of the spin tube, thereby neutralizing the sodium hydroxide.

21. (canceled)

22. A method of denaturing double-stranded DNA (dsDNA), comprising:
loading dsDNA into a tube that contains sodium hydroxide in a dry form and a neutralizing agent comprising a time-triggered coat, thereby denaturing the dsDNA; and
incubating the tube to allow for release of the neutralizing agent from the time-triggered coat, thereby neutralizing the sodium hydroxide.

23-64. (canceled)
